(19) United States
(12) Patent Application Publication
(10) Pub. No.: US 2015/0157870 A1
(43) Pub. Date: Jun. 11, 2015

(54) SHAPE CONFORMING FLEXIBLE DIELECTRIC BARRIER DISCHARGE PLASMA GENERATORS

(71) Applicants: Sameer Kalghatgi, Copley, OH (US); Tsung-Chan Tsai, Cuyahoga Falls, OH (US); Daphne Pappas Antonakas, Hudson, OH (US); Robert L. Gray, Hudson, OH (US)

(72) Inventors: Sameer Kalghatgi, Copley, OH (US); Tsung-Chan Tsai, Cuyahoga Falls, OH (US); Daphne Pappas Antonakas, Hudson, OH (US); Robert L. Gray, Hudson, OH (US)

(73) Assignee: EP TECHNOLOGIES LLC, Akron, OH (US)

(21) Appl. No.: 14/564,717

(22) Filed: Dec. 9, 2014

Related U.S. Application Data
(60) Provisional application No. 61/913,718, filed on Dec. 9, 2013.

(51) Int. Cl. A61N 1/44; A61F 7/02

(52) U.S. Cl. CPC A61N 1/44; A61F 7/02

(57) ABSTRACT

Exemplary embodiments of shape conforming dielectric barrier discharge (DBD) plasma generators are disclosed herein. One exemplary embodiment includes a flexible pad and a plurality of electrodes located in the pad within close proximity of each other, and a flexible dielectric barrier surrounding the plurality of electrodes and separating the plurality of electrodes from each other. Wherein when a high voltage pulse is applied to one or more of the plurality of electrodes, plasma is produced between a surface of the flexible pad and a portion of the body in close proximity to the flexible pad.

### RELATED APPLICATIONS

This application claims priority to and the benefits of U.S. Provisional Patent Application Ser. No. 61/913,718 filed on Dec. 9, 2013 and entitled “SHAPE CONFORMING FLEXIBLE DIELECTRIC BARRIER DISCHARGE PLASMA GENERATORS,” which is incorporated herein by reference in its entirety.

### TECHNICAL FIELD

The present invention relates generally to dielectric barrier discharge (DBD) plasma generators, and more particularly to shape conforming DBD plasma generators for use in skin and wound treatment applications.

### BACKGROUND OF THE INVENTION

Recently, it has been discovered that non-thermal atmospheric pressure plasma may be useful for skin disinfection, transdermal delivery of molecules and pharmaceuticals, wound treatment and other applications where it is desirable to treat an area of the body directly with such plasmas. Prior art devices have used fixed geometry planar DBD plasma generators, plasma jets and the like to generate plasma and to treat an area of the body, wherein the planar DBD devices and the plasma jets are moved across the area to be treated.

### SUMMARY

Exemplary embodiments of flexible shape conforming DBD plasma generators are disclosed herein. One exemplary embodiment includes a flexible pad and a plurality of electrodes located in the pad within close proximity of each other, and a flexible dielectric barrier surrounding the plurality of electrodes and separating the plurality of electrodes from each other. Wherein when a high voltage source is applied to one or more of the plurality of high voltage electrodes and the ground electrodes are connected to electrical ground potential, plasma is produced on the surface of the flexible pad.

Another exemplary embodiment of a flexible plasma generating patch for generating plasma between the flexible plasma patch and a portion of a body includes a flexible patch, conductive material located in the flexible patch, the flexible patch forming a dielectric barrier around the conductive material. A lead for placing the conductive material in circuit communication with a high voltage power source, and one or more spacers for spacing the flexible patch away from at least a portion of a body when the flexible patch is placed in proximity with a body. Wherein when a high voltage source is applied to the conductive material located in the flexible patch, plasma is produced between a surface of the flexible patch and a portion of body where the flexible patch is in close proximity with.

Another exemplary flexible plasma generating pad includes a rubber pad, a plurality of conductors located within the rubber pad, and a lead for connecting one or more of the plurality of conductors to a high voltage source. Wherein when a high voltage source is applied to one or more of the plurality of electrodes, plasma is produced on a surface of the rubber pad and a portion of a body over which the pad is either secured or is in close proximity.

Another exemplary flexible shape conforming plasma generating pads is a wearable plasma generating pad. The exemplary wearable plasma generating pad includes a flexible pad having one or more high voltage electrodes and one or more ground electrodes located within the flexible pad. The wearable plasma generating pad includes a portable power source having a first low voltage, circuitry for converting the portable power source to a second high voltage, one or more conductors for connecting second high voltage to the high voltage electrodes and one or more conductors for connecting the ground electrodes to electrical ground potential. Wherein when a high voltage source is applied to one or more of the plurality of high voltage electrodes and the ground electrodes are connected to electrical ground potential, plasma is produced on a surface of the flexible pad in close proximity to the portion of the body over which the wearable plasma generating pad is worn.

### BRIEF DESCRIPTION OF THE DRAWINGS

These and other features and advantages of the present invention will become better understood with regard to the following description and accompanying drawings in which:

FIG. 1 is a perspective view of an exemplary embodiment of a flexible shape conforming DBD generator secured around a person's wrist;

FIG. 2 is a plan view of the exemplary flexible shape conforming DBD generator of FIG. 1 in a flat position;

FIG. 3 is a perspective view of another exemplary embodiment of a flexible shape conforming DBD generator secured around a person's wrist;

FIG. 4 is a plan view of another exemplary embodiment of a flexible shape conforming DBD generator in a flat position;

FIG. 5 is a side view of the exemplary embodiment of FIG. 4;

FIG. 6 is a plan view of another exemplary embodiment of a shape conforming DBD generator in a flat position;

FIG. 7 is an exemplary embodiment of a shape conforming DBD plasma jet generator on the surface of skin; and

FIG. 8 is another exemplary embodiment of a shape conforming DBD plasma jet generator on the surface of skin.

### DETAILED DESCRIPTION

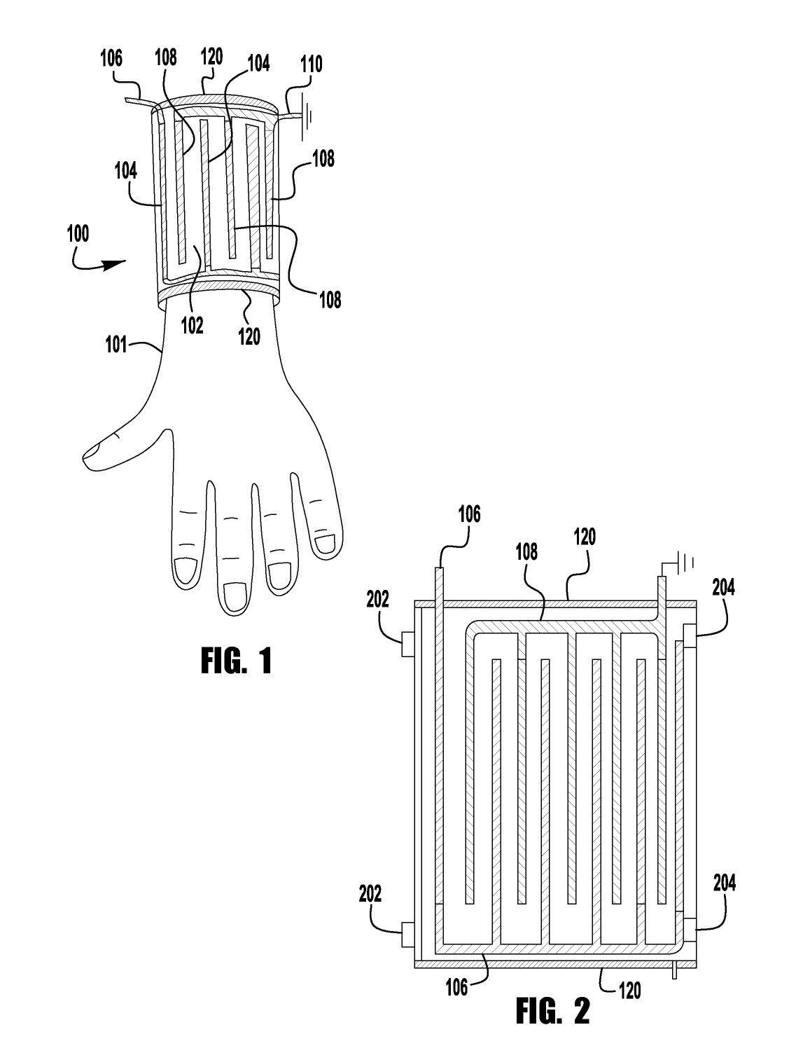

FIG. 1 is an exemplary embodiment of a flexible shape conforming DBD plasma generator 100 secured around a person's wrist 101. As used herein, shape conforming means the plasma generator 100 is not rigid and can be bent, curved, formed, shaped etc. In addition, although the flexible shape conforming DBD plasma generator 100 may conform to the surface of the object it is on, it is not required to do so. The shape conforming DBD plasma generator 100 includes a flexible pad 102. Flexible pad 102 may have any type of shape, such as, for example, square, round, rectangular, oval, or the like. In addition, in some embodiments, the flexible pad 102 may have a 3-D shape, such as, for example, a glove shape, a sock shape or the like. In some embodiments, flexible pad 102 is made of rubber, silicone, foam, urethane, gel, fabric or any other flexible material. In some embodiments, the flexible pad 102 has a hardness of less than about 55 durometers. In some embodiments, the flexible pad 102 has a hardness of less than about 45 durometers and in some embodiments less than about 30 durometers. In some embodiments, the flexible pad may include a gel with conductive properties so that when the flexible shape conforming DBD plasma generator 100 is energized, the gel warms up and heats up the treatment area and may also acts as an application medium.

Flexible pad 102 includes a plurality of high voltage electrodes 104. In some embodiments, the high voltage electrodes 104 are wires. In some embodiments, the wires are solid core wires and in some embodiments the wire may be a plurality of stranded filaments or hollow wires. Solid core or hollow wires may be bent to cause the flexible pad 102 to form a desired shape. The solid core wires tend to cause the flexible pad 102 in the desired shape. In applications where it is desired to have the flexible pad 102 conform to an object, stranded wires may be used as high voltage electrodes 104. In some embodiments, wires are insulated prior to being encapsulated in the flexible pad 102. In some embodiments, the flexible pad 102 insulates the high voltage electrodes 104. In some embodiments, the high voltage electrodes 104 are made of a flat conductive material or a ribbon material. In some embodiments, the high voltage electrodes 104 contain copper, silver, aluminum, gold, carbon nanotubes, carbon nanowires or the like, or mixtures of one or more of these conductive materials. In some embodiments, flexible pad 102 is a dielectric barrier and in some embodiments, insulation around the high voltage conductors 104 is the dielectric barrier.

The high voltage electrodes 104 are connected, or connectable to a high voltage source 106. High voltage source 106 may be a nanosecond pulsed power source, a microsecond pulsed power source, a nanosecond power source, a sinusoidal power source, RF driven power source, pulsed DC driven power source, a battery source or the like.

In addition, in some embodiments, the high voltage source 106 includes one or more batteries (not shown) and circuitry necessary to convert the low voltage to a high voltage AC source, or to a high voltage DC source, which is well known in the electrical arts. Such applications are particularly useful for wearable flexible shape conforming plasma generators, such as cuffs, socks, gloves, mittens, belts or the like.

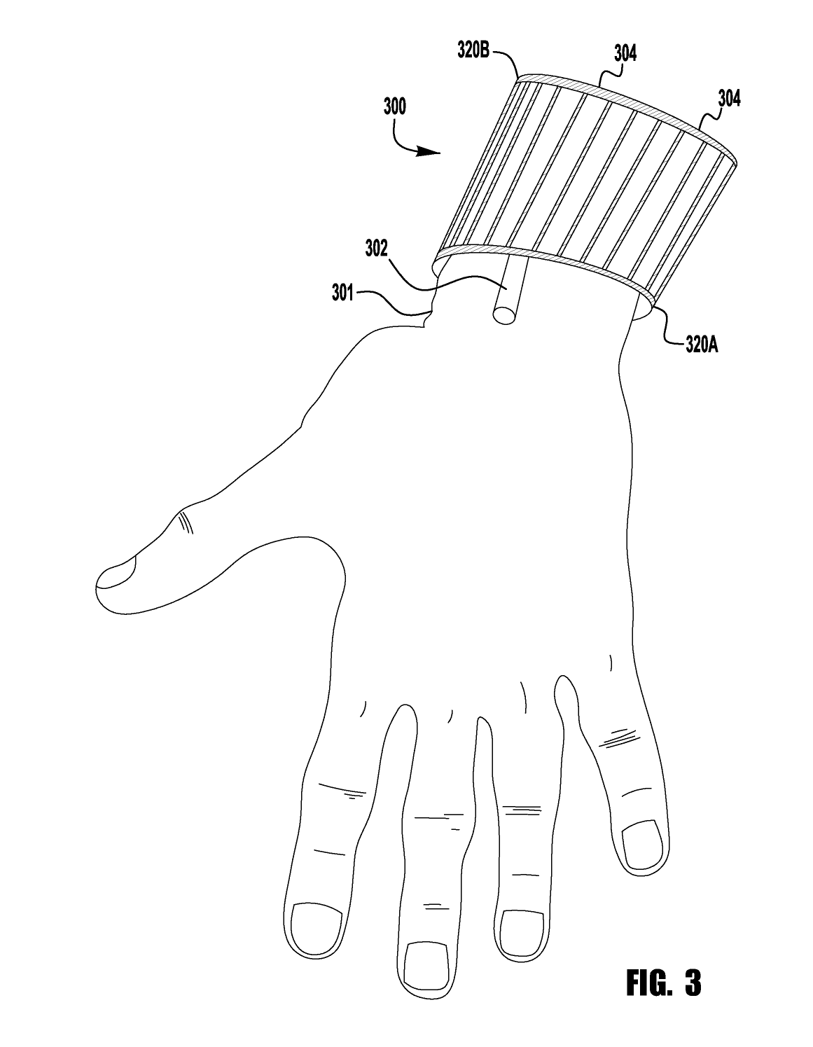

In some embodiments, shape conforming DBD plasma generator 100 includes one or more ground conductors 108. The one or more ground conductors 108 may be any conductive element, such as, for example, the conductive elements described above with respect to the high voltage electrodes 104. In the exemplary embodiment illustrated in FIGS. 1 and 2, the flexible pad 102 has alternating high voltage electrodes 104 and ground conductors 108. In some embodiments, the high voltage electrodes 104 span substantially across the length of the shape conforming DBD plasma generator 100. In some embodiments, high voltage electrodes 104 are separated by ground conductors 108. In some embodiments, ground conductors are not used. In such an embodiment, the object to be treated with plasma is grounded, and the object serves as the ground conductor 108.

In some embodiments, the high voltage electrodes 104 are separated from the ground electrodes 108 by between about 2 millimeters and about 5 millimeters. In embodiments without ground electrodes, the high voltage electrodes 104 are spaced apart from one another by a distance of between about 2 millimeters and about 5 millimeters.

As illustrated in FIG. 2, the shape conforming DBD plasma generator 100 may include connectors 202, 204 to connect the shape conforming DBD plasma generator 100 to an object, such as a person's wrist 101. Connectors 202, 204 may be, for example, Velcro® hook and loop fasteners, one or more strings, tapes, etc.

The exemplary flexible shape conforming DBD plasma generator 100 of FIGS. 1 and 2 includes one or more spacers 120. Spacers 120 may be used to elevate flexible shape conforming DBD plasma generator 100 away from a surface creating a space between flexible pad 102 and the object allowing for plasma to be generated in the space. In some embodiments, spacers 120 are between about 1 millimeter and about 3 millimeter which create a space between the bottom of flexible pad 102 and the object of between about 1 millimeter and about 3 millimeters.

In some embodiments, the spacers 120 include an adhesive on the bottom so that the spacers 120 adhere to the object. In some embodiments the spacers 120 form a seal against the object creating a sealed environment between the bottom of the flexible pad 120 and the object.

FIG. 3 illustrates another exemplary embodiment of a shape conforming DBD plasma generator 300. Shape conforming DBD plasma generator 300 is similar to shape conforming DBD plasma generator 100 and the connections to the high voltage source and ground are not illustrated. Shape conforming DBD plasma generator 300 includes a first spacer 320A and a second spacer 320B. First spacer 320A and second spacer 320B form a seal around the wrist 301. First spacer 320A includes a gas inlet 302. Gas inlet 302 may be connected to a suitable gas supply to generate electric field, reactive species and/or plasmas with different temperatures. Some exemplary gases that may be used to generate non-thermal plasma are inert gasses, such as, for example, He, Ar, Ne, Xe and/or the like, combinations thereof, air, or mixtures of inert gases with small percentage (0.5%-20%) of other gases such as O₂ and N₂. In addition, mixtures of inert gases with vaporized liquids including water, dimethyl sulfoxide (DMSO), ethanol, isopropyl alcohol, n-butanol, with or without additives and the like may be used. Accordingly, a desired gas/vapor may be forced between the shape conforming DBD plasma generator 300 and the object to create different reactive species for different types of treatment. In some embodiments, spacer 320B includes one or more apertures 304 that allow gas/vapor to flow out from under the shape conforming DBD plasma generator 300.

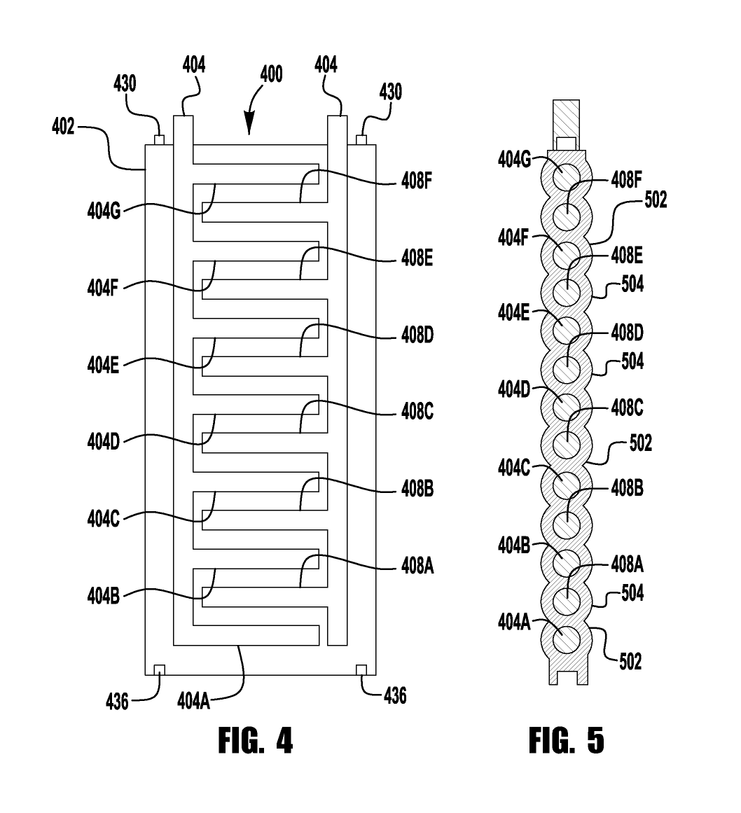

FIGS. 4 and 5 illustrate another exemplary embodiment of a flexible shape conforming DBD plasma generator 500. Flexible shape conforming DBD plasma generator 400 includes a flexible pad 402. Flexible pad 402 includes a high voltage electrode 404 that extends along a length of the flexible pad 402 and has extensions 404A, 404B, 404C, 404D, 404E, 404F and 404G that extend along a width of the flexible pad 402. High voltage electrode 404 is connectable to a high voltage source (not shown) as described above.

Similarly, flexible pad 402 includes a ground electrode 408 that extends along a length of the flexible pad 402 and has extensions 408A, 408B, 408C, 408D, 408E and 408F that extend along a width of the flexible pad 402. Ground electrode 408 is connectable to ground. Flexible pad 402 includes connectors 430 and 434, which may be used to secure flexible pad 402 to an object. As noted above, in some embodiments, the high voltage electrodes 404A-404G are separated from the ground electrodes 408A-408F by between about 1 millimeter and about 5 millimeters.

As illustrated in FIG. 5, the bottom surface 500 of flexible pad 402 is undulated having upper surfaces 502 and lower surfaces 504. Accordingly, when placed on an object, the lower surfaces 504 contact the object and there is a space between the object and the upper surface 502 where plasma gas may form. In some embodiments, spacers (not shown) may be included to increase the space, and to create space between the lower surface 504 and the object.

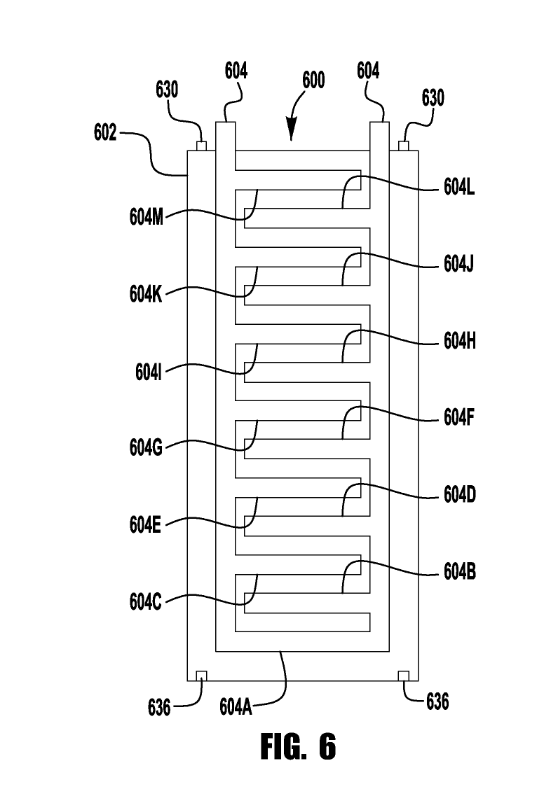

FIG. 6 illustrates another exemplary embodiment of a shape conforming DBD plasma generator 600. Shape conforming DBD plasma generator 600 is similar to shape conforming DBD plasma generator 500 except shape conforming DBD plasma generator 600 does not include grounding electrodes. The object, such as, for example, a body part, is grounded. Flexible shape conforming DBD plasma generator 600 includes a flexible pad 602. Flexible pad 602 includes a high voltage electrode 604 that extends along a length of the flexible pad 602 and has extensions 604A, 604B, 604C, 604D, 604E, 604F, 604G, 604H, 6041, 604J, 604K, 604L and 604M that extend along a width of the flexible pad 602. In this embodiment the extensions are connected together on their second end by electrode 605, which extends along the length of flexible pad 602. High voltage electrode 604 is connectable to a high voltage source (not shown) as described above.

During operation, a flexible shape conforming DBD plasma generator 100, 300, 400, 600 is placed on a body part. Flexible shape conforming DBD plasma generator 100, 300, 400, 600 either self conforms to the shape of the body part, or is molded to a desired shape by an operator. In some embodiments, the shape conforming DBD plasma generator 100, 300, 400, 600 is secured to the body part by, for example adhesive. In some embodiments, the shape conforming DBD plasma generator 100, 300, 400, 600 includes an inflatable portion similar to a blood pressure cuff, which is wrapped around a body part and inflated to locate the shape conforming DBD plasma generator 100, 300, 400, 600 in the proper location.

A high voltage is placed on high voltage electrode 104, 404, 604 and grounding electrodes 108, 408 are grounded, or the body part is grounded. Plasma is formed by the application of the high voltage and is used to treat the desired body part. The treatment time may be for a few seconds, few minutes or for longer periods of time. In some embodiments, the shape conforming DBD plasma generator 100, 300, 400, 600 is left on the body part for an extended period of time. A portable high voltage source, such as, for example, one or more batteries may be used in such situations. In some embodiments, the portable high voltage source is connected to a wearable flexible shape conforming plasma generator. In such embodiments, multiple treatments of shorter duration may be beneficial and the portable high voltage source may allow a person the freedom to move around or leave the treatment area.

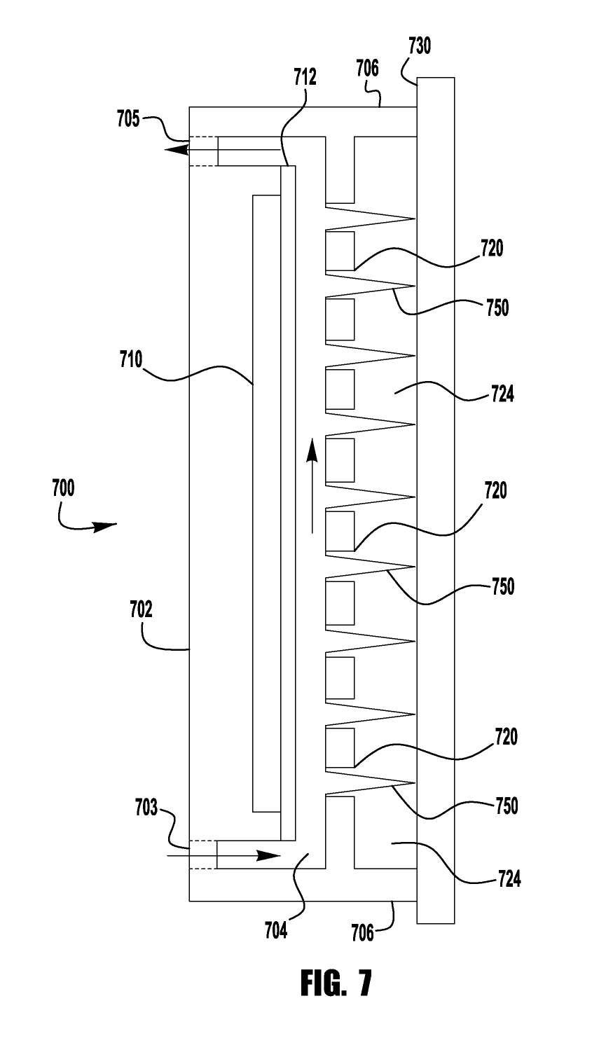

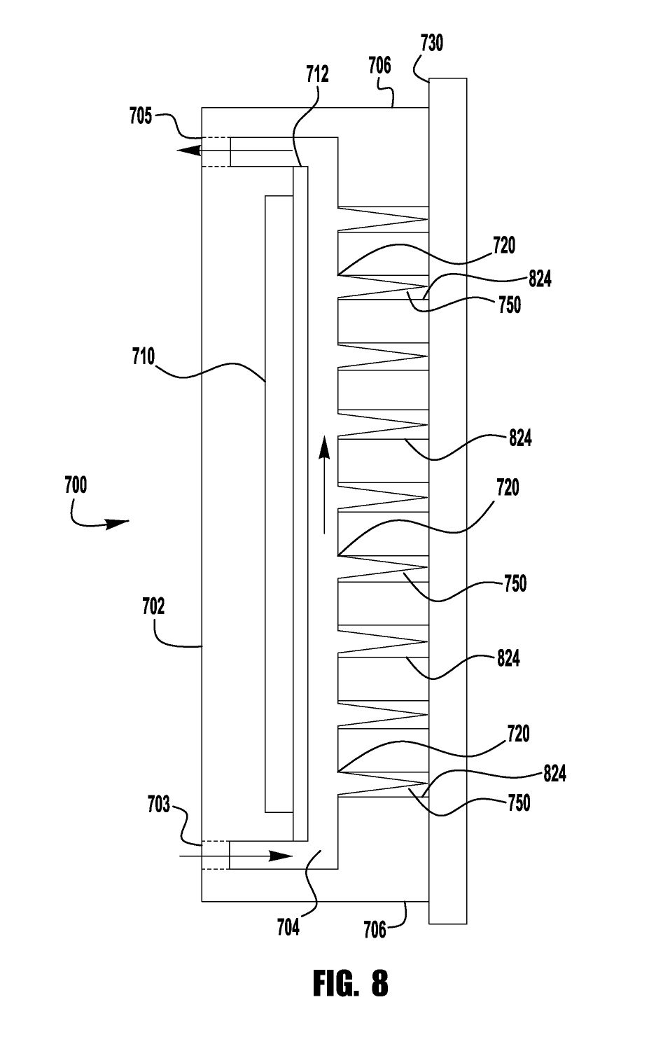

FIGS. 7 and 8 are exemplary embodiments of flexible DBD plasma jet pads 700, 800. Many of the above features described above with respect to FIGS. 1-6 may also be used with the exemplary embodiments using flexible DBD plasma jet pads 700, 800, such as for example, an adhesive may be located on the area that contacts the surface of the skin to adhere the flexible DBD plasma jet pads 700, 800 to the skin. Accordingly, the features identified above may not be re-identified with respect to FIGS. 7 and 8.

The exemplary flexible DBD plasma jet pad 700 is made of a flexible material 702, such as for example, rubber, silicone or the like. Flexible DBD plasma jet pad 700 includes a gas inlet 703. Suitable gases for use with flexible DBD plasma jet pad 700 include, for example, air, helium, nitrogen and the like. The gas flows in through the inlet 703, through passage 704 and out of gas outlet 705.

A flexible electrode 710 is located in the flexible material 702. Located between passage 704 and the flexible electrode 710 is a dielectric barrier 712, such as, for example, a quartz dielectric. In some embodiments, the dielectric barrier 712 is made of the same material as the flexible material 702. Flexible DBD plasma jet pad 700 includes spacers 706 for spacing the flexible DBD plasma jet pad 700 away from the surface of the skin 730. In some embodiments, skin 730 is grounded. In some embodiments, skin 730 has a floating ground. An adhesive material (not shown) may be included on the bottom of the spacers to cause the flexible pad to self adhere to the skin 730.

Spacers 706 form a treatment cavity 724 that surrounds a desired treatment area on the surface of the skin 730. A plurality of apertures 720 between passage 704 and treatment cavity 724 allow plasma jets 750 to pass into the treatment cavity 724 and contact the surface of the skin 730. In some embodiments, the plasma jets do not contact the skin. The plasma jets pass through the apertures 720 due to the surface of the skin 730 due to the difference in potential.

FIG. 8 illustrates another embodiment of exemplary flexible DBD plasma jet pad 800. Flexible DBD plasma jet pad 800 is similar to flexible DBD plasma jet pad 700 and common elements are illustrated with common designators. One difference is that flexible DBD plasma jet 700 includes a single treatment cavity 724, while flexible DBD plasma jet pad 800 includes a plurality of treatment cavities 824.

In some embodiments, the treatment cavities 724, 824 have a circular shape, a square shape, and octagonal shape, a honeycomb shape, an elongated shape, combinations of shapes or the like. In all of the embodiments equipped with a gas inlet, drugs or other additives, may added to the gas inlet to be applied to the skin.

In some embodiments, the flexible DBD plasma generators include a quick connector (not shown) that allows the high voltage source to be connected to and disconnected from the flexible DBD plasma generators. In some embodiments, the quick connector includes a sealing member that seals the high voltage electrode on the flexible pad so that flexible pad may be submerged in water when not connected to the high voltage source. Thus, the flexible pads may be left on a patient. Accordingly, a flexible pad may be adhered to a patient and then for subsequent treatments, the patient need only stop into the treatment center periodically and be connected to the power source to receive treatments without the step of having to have a flexible pad adhered to and removed from the patient each time they show up for treatment.

In some embodiments, the flexible plasma generators described herein may be cut to size. In such embodiments, the electrodes may contain frangible portions that may be broken off so that the ends of the electrodes are located within the flexible pad, and in some embodiments, sealed off from contact by the material making up the flexible pad once the flexible pad is cut to size. In some embodiments, the flexible pads are modular flexible pads and the modular flexible pads may be joined or fitted together to form a desired shape or size to cover a treatment area.

In some embodiments, the flexible material is breathable to allow moisture between the skin and the flexible pad to pass out through the flexible pad, but prevents moisture from passing through the flexible pad to the area between the flexible pad and the skin.

In another exemplary embodiment, the flexible pad includes a two-dimensional corona array (not shown). As described above, the device contains a flexible insulating or dielectric material with an embedded conducting material. Connected to the conducting material is an array of corona needles (not shown) in a two dimensional array format. A spacer is included to maintain a gap of 1-3 mm between the needles and a surface, such as, for example, skin. The device is flexible, as shown and described above, so it may wrap around a surface, such as, for example, a wrist, or or lay flat on a larger surface, such as a back or stomach. In some embodiments, the tips of the needles are hollow and allow the delivery of drugs/vaccines before, during or after plasma treatment. In some embodiments, the flexible pad and array is adjustable in size. For example, the flexible pad/array may be manufactured as a 10″×10″ block containing 100 1″×1″ corona arrays with perforations between. The user can tear off or cut off as large or as small of an array depending on the treatment required; for example 2″×2″, etc. In such an embodiment, the whole array may be conductive no matter how many or how few arrays are used.

In some embodiments, the needles are about 0.2-3 mm long with a spacing of 0.2 to 1 mm between the needles. The gap may be controlled to maintain a spacing of about 1 to about 3 mm between the needles and the surface, such as, for example, skin. The conductive material and the needles may be made of any conductive metal or material. The needles may be rigid or flexible. The flexible material that makes up the flexible pad and contains the conductive material may be an insulating material or dielectric material. The power supply may be a nanosecond or a picosecond pulsed power supply, and the time of treatment may be a few seconds to minutes. The pulse duration may range from ˜100 μs to 40 ns. The applied voltage may range from 3-30 kV. Pulse repetition frequency may be 1-1000 Hz.

The shape conforming flexible dielectric barrier discharge plasma generators disclosed herein may be used to promote wound healing, to sterilize a treatment area, to apply antimicrobials, such as an antimicrobial cream. In the case of promoting wound healing, in some embodiments, nitrous oxide, nitrogen or air may be used for generating plasma.

The flexible dielectric barrier discharge plasma generators may be used for chronic wound care. In some embodiments, the flexible dielectric barrier discharge plasma generators are used below bandages. Although some embodiments are shown and described as being applied directly to the surface of the skin, in some embodiments, the flexible dielectric barrier discharge plasma generators are applied over bandages.

In some embodiments, the flexible pads are applied during pre-operation to sterilize a surgical site. In some embodiments, the flexible pads are applied to a surgical site post-operation to sterilize the surgical site. In some embodiments, the flexible pads are applied both pre-operation and post-operation.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and illustrative examples shown and described. Accordingly, departures may be made from such details without departing from the spirit or scope of the applicant's general inventive concept.

We claim:

1. A flexible shape conforming DBD plasma generator for conforming to the shape of a body comprising:
a flexible pad;
a plurality of electrodes located in the pad within close proximity of each other; and
a flexible dielectric barrier surrounding the plurality of electrodes and separating the plurality of electrodes from each other;
wherein when a high voltage source is applied to one or more of the plurality of electrodes plasma is produced on a surface of the flexible pad.

2. The flexible shape conforming DBD plasma generator of claim 1 wherein the flexible pad is also the flexible dielectric barrier.

3. The flexible shape conforming DBD plasma generator of claim 1 further comprising a high voltage source in circuit communication with the plurality of electrodes for providing high voltage pulses to one or more of the plurality of electrodes to generate a plasma field.

4. The flexible shape conforming DBD plasma generator of claim 3 wherein the high voltage source comprises a battery.

5. The flexible shape conforming DBD plasma generator of claim 1 further comprising a spacer for spacing the flexible away from a body portion.

6. The flexible shape conforming DBD plasma generator of claim 1 further comprising one or more connectors.

7. The flexible shape conforming DBD plasma generator of claim 1 wherein one or more of the plurality of electrodes are grounded.

8. The flexible shape conforming DBD plasma generator of claim 1 wherein the plurality of electrodes are high voltage electrodes and the surface of an object provides a floating ground and plasma is generated between the high voltage electrodes and the floating ground.

9. The flexible shape conforming DBD plasma generator of claim 1 wherein at least one of the electrodes has one or more flat surfaces.

10. The flexible shape conforming DBD plasma generator of claim 1 wherein the flexible pad comprising a conductive gel that is heated by one or more of the plurality of electrodes to warm the flexible pad.

11. The flexible shape conforming DBD plasma generator of claim 1 wherein the plurality of electrodes are separated by less than 3 millimeters.

12. The flexible shape conforming DBD plasma generator of claim 1 wherein the plurality of electrodes are separated by between about 2 and about 5 millimeters.

13. The flexible shape conforming DBD plasma generator of claim 1 wherein the flexible pad is configured to provide a gap away from at least a portion of the flexible pad and a body part.

14. The flexible shape conforming DBD plasma generator of claim 13 wherein the gap is less than about 5 millimeters.

15. The flexible shape conforming DBD plasma generator of claim 1 further comprising a gas inlet.

16. A flexible plasma generating patch for generating plasma between the flexible plasma patch and a portion of a body comprising:
a flexible patch;
conductive material located in the flexible patch;
the flexible patch forming a dielectric barrier around the conductive material;
a lead for placing the conductive material in circuit communication with a high voltage power source;
one or more spacers for spacing the flexible patch away from at least a portion of a body when the flexible patch is placed in proximity with a body;
wherein when a high voltage pulse is applied to the conductive material located in the flexible patch, plasma is produced between a surface of the flexible patch and the portion of the body in close proximity to the flexible patch.

17. The flexible patch of claim 16 wherein the one or more spacers are protrusions on the flexible patch.

18. The flexible patch of claim 16 wherein the one or more spacers are crests of an undulated surface of the flexible patch.

19. A flexible plasma generating pad comprising:
a rubber pad;
a plurality of conductors located within the rubber pad;
a lead for connecting one or more of the plurality of conductors to a high voltage sources;
wherein when a high voltage pulse is applied to one or more of the plurality of electrodes, plasma is produced between a surface of the rubber pad and a portion of a body in close proximity to the rubber pad.

20. The flexible plasma generating pad of claim 19 wherein the plurality of conductors are malleable and may be bent to conform to a desired shape and wherein the plurality of conductors hold the rubber pad in the desired shape.

21. The flexible plasma generating pad of claim 19 further comprising a high voltage source connected to the lead.

22. The flexible plasma generating pad of claim 21 wherein the high voltage source comprises a battery and a transformer.

23. The flexible shape conforming DBD plasma generator of claim 19 further comprising a gas inlet.
